(19) United States
(12) Patent Application Publication
(10) Pub. No.: US 2009/0191535 A1
(43) Pub. Date: Jul. 30, 2009

(54) Method of assessing metastatic carcinomas from circulating endothelial cells and disseminated tumor cells

(76) Inventors: Mark Carle Connelly, Doylestown, PA (US); Gerald V. Doyle, Radnor, PA (US); Galla Chandra Rao, Princeton Junction, NJ (US); Leon W.M.M. Terstappen, Amsterdam (NL)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/004,805

(22) Filed: Dec. 22, 2007

(51) Int. Cl. A01N 1/02; C12Q 1/02

(52) U.S. Cl. 435/2; 435/29

(57) ABSTRACT

A method for assessing cancer in test subjects is described based upon enumeration of circulating endothelial cells and/or disseminated tumor cells in a test subject. This method is used to quantify disseminated tumor cells. Correlations with circulating tumor cells provides prognostic information with high accuracy in assessing the risk of recurrence in patients with primary breast cancer.

### CROSS-REFERENCE TO RELATED APPLICATIONS

This is a non-provisional application which claims priority to U.S. Provisional Applications 60/686,701, filed Jun. 2, 2005, and 60/686,705, filed Jun. 2, 2005. This application also claims priority to U.S. application Ser. No. 11/202,875, filed Aug. 12, 2005. Each of the aforementioned applications is incorporated in full by reference herein.

### BACKGROUND

1. Field of the Invention

The invention relates generally to cancer prognosis and survival in metastatic cancer patients, based on the presence of morphologically intact circulating cancer cells (CTC) in blood. More specifically, diagnostic methods, reagents and apparatus are described that correlate the presence of circulating cancer cells in 7.5 ml of blood of metastatic breast cancer patients with time to disease progression and survivability. Circulating tumor cells are determined by highly sensitive methodologies capable of isolating and imaging 1 or 2 cancer cells in approximately 5 to 50 ml of peripheral blood, the level of the tumor cell number and an increase in tumor cell number during treatment are correlated to the time to progression, time to death and response to therapy.

2. Background Art

Despite efforts to improve treatment and management of cancer patients, survival in cancer patients has not improved over the past two decades for many cancer types. Accordingly, most cancer patients are not killed by their primary tumor, but they succumb instead to metastases: multiple widespread tumor colonies established by malignant cells that detach themselves from the original tumor and travel through the body, often to distant sites. The most successful therapeutic strategy in cancer is early detection and surgical removal of the tumor while still organ confined. Early detection of cancer has proven feasible for some cancers, particularly where appropriate diagnostic tests exist such as PAP smears in cervical cancer, mammography in breast cancer, and serum prostate specific antigen (PSA) in prostate cancer. However, many cancers detected at early stages have established micrometastases prior to surgical resection. Thus, early and accurate determination of the cancer's malignant potential is important for selection of proper therapy.

Optimal therapy will be based on a combination of diagnostic and prognostic information. An accurate and reproducible diagnostic test is needed to provide specific information regarding the metastatic nature of a particular cancer, together with a prognostic assessment that will provide specific information regarding survival.

A properly designed prognostic test will give physicians information about risk and likelihood of survival, which in turn gives the patient the benefit of not having to endure unnecessary treatment. Patient morale would also be boosted from the knowledge that a selected therapy will be effective based on a prognostic test. The cost savings associated with such a test could be significant as the physician would be provided with a rationale for replacing ineffective therapies. A properly developed diagnostic and prognostic data bank in the treatment and detection of metastatic cancer focusing on survival obviously would provide an enormous benefit to medicine (U.S. Pat. No. 6,063,586).

If a primary tumor is detected early enough, it can often be eliminated by surgery, radiation, or chemotherapy or some combination of those treatments. Unfortunately, the metastatic colonies are difficult to detect and eliminate and it is often impossible to treat all of them successfully. Therefore from a clinical point of view, metastasis can be considered the conclusive event in the natural progression of cancer. Moreover, the ability to metastasize is a property that uniquely characterizes a malignant tumor.

### Soluble Tumor Antigen:

Based on the complexity of cancer and cancer metastasis and the frustration in treating cancer patients over the years, many attempts have been made to develop diagnostic tests to guide treatment and monitor the effects of such treatment on metastasis or relapse.

One of the first attempts to develop a useful test for diagnostic oncology was the formulation of an immunoassay for carcinoembryonic antigen (CEA). This antigen appears on fetal cells and reappears on tumor cells in certain cancers. Extensive efforts have been made to evaluate the usefulness of testing for CEA as well as many other “tumor” antigens, such as prostate specific antigen (PSA), CA 15.3, CA 125, prostate-specific membrane antigen (PSMA), CA 27.29, p27 found in either tissue samples or blood as soluble cellular debris. These and other debris antigens are thought to be derived from destruction of both circulating and non-circulating tumor cells, and thus their presence may not always reflect metastatic potential, especially if the cells rupture while in an apoptotic state.

Additional tests used to predict tumor progression in cancer patients have focused upon correlating enzymatic indices like telomerase activity in biopsy-harvested tumor samples with an indication of an unfavorable or favorable prognosis (U.S. Pat. No. 5,693,474; U.S. Pat. No. 5,639,613). Assessing enzyme activity in this type of analysis can involve time-consuming laboratory procedures such as gel electrophoresis and Western blot analysis. Also, there are variations in the signal to noise and sensitivity in sample analysis based on the origin of the tumor. Despite these shortcomings, specific soluble tumor markers in blood can provide a rapid and efficient approach for developing a therapeutic strategy early in treatment. Increased HER-2/neu results in decreased response to hormone therapy, and is a significant prognostic factor in predicting responses to hormone receptor-positive metastatic breast cancer. Thus in malignancies where the HER-2/neu oncogene product is associated, methods have been described to monitor therapy or assess risks based on elevated levels (U.S. Pat. No. 5,876,712). However in both cases, the base levels during remission, or even in healthy normals, are relatively high and may overlap with concentrations found in patients, thus requiring multiple testing and monitoring to establish patient-dependent baseline and cut-off levels.

In prostate cancer, PSA levels in serum have proven to be useful in early detection. When used with a follow-up physical examination (digital rectal exam) and biopsy, the PSA test has improved detection of prostate cancer at an early stage when it is best treated.

However, PSA or the related PSMA testing leaves much to be desired. For example, elevated levels of PSA weakly correlate with disease stage and appear not to be a reliable indicator of the metastatic potential of the tumor. This may be due in part to the fact that PSA is a component of normal prostate tissue and benign prostatic hyperplasia (BHP) tissue. Moreover, approximately 30% of patients with alleged localized prostate cancer and corresponding low serum PSA concentrations, may have metastatic disease (Moreno et al., Cancer Research, 52:6110 (1992)).

The aforementioned studies do not provide for consistent data with a long follow-up period or at a satisfactory specificity. Accordingly, these efforts have proven to be somewhat futile as the appearance of mRNA for antigens in blood have not been generally predictive for most cancers and are often detected when there is little hope for the patient.

Using Kaplan-Meier type analysis, disease free survival of patients with positive CEA mRNA in post-operative blood was significantly shorter than in cases that were negative for CEA mRNA. These results suggest that tumor cells were shed into the bloodstream (possibly during surgical procedures or from micro metastases already existing at the time of the operation), and resulted in poor patient outcomes in patients with colorectal cancer. The sensitivity of this assay provided a reproducibly detectable range similar in sensitivity to conventional RT-PCR. As mentioned, these detection ranges are based on unreliable conversions of amplified product to the number of tumor cells. The extrapolated cell count may include damaged CTC incapable of metastatic proliferation. Further, PCR-based assays are limited by possible sample contamination, along with an inability to quantify tumor cells. Most importantly, methods based on PCR, flowcytometry, cytoplasmic enzymes and circulating tumor antigens cannot provide essential morphological information confirming the structural integrity underlying metastatic potential of the presumed CTC and thus constitute functionally less reliable surrogate assays than the highly sensitive imaging methods embodied, in part, in this invention.

### Assessment of Intact Tumor Cells in Cancer Detection and Prognosis:

Detection of intact tumor cells in blood provides a direct link to recurrent metastatic disease in cancer patients who have undergone resection of their primary tumor. Unfortunately, the same spreading of malignant cells continues to be missed by conventional tumor staging procedures. Recent studies have shown that the presence of a single carcinoma cell in the bone marrow of cancer patients is an independent prognostic factor for metastatic relapse (Diel I J, Kaufman M, Goerner R, Costa S D, Kaul S, Bastert G. Detection of tumor cells in bone marrow of patients with primary breast cancer: a prognostic factor for distant metastasis. J Clin Oncol, 10:1534-1539, 1992). But these invasive techniques are deemed undesirable or unacceptable for routine or multiple clinical assays compared to detection of disseminated epithelial tumor cells in blood.

An alternative approach incorporates immunomagnetic separation technology and provides greater sensitivity and specificity in the unequivocal detection of intact circulating cancer cells. This simple and sensitive diagnostic tool, as described (U.S. Pat. No. 6,365,362; U.S. Pat. No. 6,551,843; U.S. Pat. No. 6,623,982; U.S. Pat. No. 6,620,627; U.S. Pat. No. 6,645,731; WO 02/077604; WO03/065042; and WO 03/019141) is used in the present invention to correlate the statistical survivability of an individual patient.

Using this diagnostic tool, a blood sample from a cancer patient (WO 03/018757) is incubated with magnetic beads, coated with antibodies directed against an epithelial cell surface antigen as for example EpCAM. After labeling with anti-EpCAM-coated magnetic nanoparticles, the magnetically labeled cells are then isolated using a magnetic separator. The immunomagnetically enriched fraction is further processed for downstream immunocytochemical analysis or image cytometry, for example, in the CellTracks® System (Immunicon Corp., PA). The magnetic fraction can also be used for downstream immunocytochemical analysis, RT-PCR, PCR, FISH, flowcytometry, or other types of image cytometry.

The CellTracks® System utilizes immunomagnetic selection and separation to highly enrich and concentrate any epithelial cells present in whole blood samples. The captured cells are detectably labeled with a leukocyte specific marker and with one or more tumor cell specific fluorescent monoclonal antibodies to allow identification and enumeration of the captured CTC's as well as unequivocal instrumental or visual differentiation from contaminating non-target cells. At an extraordinary sensitivity of 1 or 2 epithelial cells per 7.5-30 ml of blood, this assay allows tumor cell detection even in the early stages of low tumor mass. The embodiment of the present invention is not limited to the Cell Spotter® System, but includes any isolation and imaging protocol of comparable sensitivity and specificity.

### Assessment of Intact CEC in Cancer Detection and Prognosis:

The human vasculature is integral to a human's health and knowledge of its general and local condition is of great importance. Endothelial cells line the luminal surface of blood vessels and are believed to be involved with the pathogenesis of multiple disease conditions including cancer, cardiovascular diseases, autoimmune diseases, infectious diseases, and various benign conditions. Endothelial cells can detach from their monolayer and end up in the circulation. The cause for the detachment, fate and role of these circulating endothelial cells (CECs) is not yet understood. The enumeration and characterization of CECs may however offer a unique opportunity to study the vasculature and improve our understanding of a variety of disease processes. Elevation of the number of circulating endothelial cells have been observed in a variety of pathological conditions such as cardiovascular diseases, inflammation, infection, autoimmune disease, and cancer. In cancer CECs may increase in the circulation due to active angiogenesis or vascular damage due to tumor degeneration or as a sight effect of therapy. The great variation in the reported ranges of CECs from 1 to 1000 per mL makes the interpretation of these reported results quite difficult if not impossible. The large variation in the definition of CECs and the technologies used to measure the CECs are the main contributors. In addition little attention is paid to the characterization of the assays used to enumerate CECs. We developed an automated sample preparation system to immunomagnetically select CD146 expressing cells and fluorescently label these cells with DAPI, CD45 and CD105 from 4 mL of blood. The CECs defined as DAPI+, CD146+, CD105+, CD45− cells were identified and enumerated with a semi-automated fluorescence microscope. After a thorough characterization of the CEC assay, CECs were enumerated under different conditions in healthy individuals and than compared to those found in patients with metastatic carcinomas.

Currently available prognostic protocols have not demonstrated a reliable means for correlating circulating endothelial cells (CEC) and/or disseminated tumor cells (DTC) to predict progression free- or overall survival in patients with cancers such as metastatic breast cancer (MBC). Thus, there is a clear need for accurate detection of these cells in assessing metastatic potential, not only in MBC but in metastatic cancers in general. Moreover, this need is accentuated by the need to select the most effective therapy for a given patient.

### SUMMARY OF THE INVENTION

The present invention is a method and means for cancer prognosis, incorporating diagnostic tools in assessing time to disease progression, survival, and response to therapy based upon the absolute number, change, or combinations of both of circulating endothelial cells (CEC), circulating tumor cells (CTC) or disseminated tumor cells in bone (DTC) from patients with metastatic cancer. The system immunomagnetically concentrates the cells, fluorescently labels the cells, identifies and quantifies for positive enumeration. The statistical analysis of the cell count predicts survival.

More specifically, the present invention provides the apparatus, methods, and kits for assessing patient survival, the time to disease progression, and response to therapy in MBC. The accurate cell enumeration provides a basisi for prediction of survival, based upon a threshold comparison of the number of cells in blood.

### BRIEF DESCRIPTION OF THE DRAWINGS

FIG. 1: Classification of endothelial cell candidates. Seven rows of thumbnails of cell candidates from a blood sample. From right to left the columns show the DAPI, CD105 PE, DiOC16 and CD45 APC staining and a composite of DAPI and CD105 (green) staining. Row 1 and 2 show a DiOC16 prelabeled HUVEC cell staining with DAPI, CD105 and lacking CD45. Row 3, 4 and 5 show an endothelial cell staining with DAPI and CD105 but lacking CD45. Note that in row 4 the endothelial cell is surrounded by two leukocytes. Row 6 and 7 show leukocytes that express CD105. The checks in the boxes indicate the cell type and are tabulated by the software.

FIG. 2: Gallery of CEC images. Nuclear staining of DAPI with CD105 staining.

FIG. 3:

Panel A shows the correlation between the number of DiOC16 prelabeled HUVEC cells spiked in 4 mL blood aliquots and the number of HUVEC cells detected after sample processing and analysis. Correlation Coefficient is R²=0.99 with a slope of 0.72 (95% CI 0.68 to 0.76) and an intercept of 5.09 (95% CI −16 to 27).
Panel B shows the assay efficiency at low spike levels, 2-26 in 4 mL of blood.

FIG. 4: Assay imprecision tested over a 20 day period at a low (48) and high (1014) cell spike level in 4 mL of blood

FIG. 5: Assay reproducibility. Panel A shows the correlation between two operators blinded to each others results analyzing the same date from 100 samples. Correlation Coefficient is R²=0.99 with a slope of 1.03 (95% CI 1.01 to 1.05) and an intercept of 0.28 (95% CI −0.72 to 1.29). The Bland-Altman plot of this data is shown in Panel B. Panel C shows the correlation between CECs enumerated from two 4 mL aliquots of blood from 72 samples. Correlation Coefficient is R²=0.97 with a slope of 0.97 (95% CI 0.89 to 1.05) and an intercept of 3.55 (95% CI −0.30 to 7.40). The Bland-Altman plot of this data is shown in Panel D.

FIG. 6: Blood draw and CEC counts. Panel A shows the correlation between the CECs counts from the first and second blood draw tube after vena puncture from 66 samples. Correlation Coefficient is R²=0.48 with a slope of 0.53 (95% CI 0.39 to 0.68) and an intercept of 4.64 (95% CI −3.31 to 12.59). Four data points were outside 3 SD and removed from the analysis. The Bland-Altman plot of this data is shown in Panel B. Panel C shows the correlation between CECs counts from the second and third blood draw tube after vena puncture from 100 samples. Correlation Coefficient is R²=0.60 with a slope of 0.61 (95% CI 0.51 to 0.71) and an intercept of 6.53 (95% CI −2.82 to 10.24). The Bland-Altman plot of this data is shown in Panel D.

FIG. 7: Prevalence of CECs in 4 mL of blood from 167 healthy individuals and 206 patients with metastatic carcinomas. 50 breast cancer patients, 49 colorectal cancer patients, 35 lung cancer patients, 48 prostate cancer patients and a group of other carcinomas consisting of 8 ovarian/pancreatic, 3 renal, 2 bladder, 2 thyroid, 2 gastric, and 1 breast/colon, colon/prostate, esophageal, gastric, carcinoid tumor, squamous cell, tongue, and mandibular cancer patients.

### DETAILED DESCRIPTION OF THE INVENTION

An accepted method for collecting circulating tumor cells combines immunomagnetic enrichment technology, immunofluorescent labeling technology with an appropriate analytical platform after initial blood draw. The associated test has the sensitivity and specificity to detect rare cells in a sample of whole blood and to investigate their role in the clinical course of the disease in malignant tumors of epithelial origin. From a sample of whole blood, rare cells are detected with a sensitivity and specificity to allow them to be collected and used in the diagnostic assays of the invention, namely predicting the clinical course of disease in malignant tumors.

With this technology, circulating tumor cells (CTC) have been shown to exist in the blood in detectable amounts. This created a tool to investigate the significance of cells of epithelial origin in the peripheral circulation of cancer patients (Racila E., Euhus D., Weiss A. J., Rao C., McConnell J., Terstappen L. W. M. M. and Uhr J. W., Detection and characterization of carcinoma cells in the blood, Proc. Natl. Acad. Sci. USA, 95:4589-4594 (1998)). This study demonstrated that these blood-borne cells might have a significant role in the pathophysiology of cancer. Having a detection sensitivity of 1 epithelial cell per 5 ml of blood, the assay incorporates immunomagnetic sample enrichment and fluorescent monoclonal antibody staining followed by flowcytometry for a rapid and sensitive analysis of a sample. The results show that the number of epithelial cells in peripheral blood of eight patients treated for metastatic carcinoma of the breast correlate with disease progression and response to therapy. In 13 of 14 patients with localized disease, 5 of 5 patients with lymph node involvement and 11 of 11 patients with distant metastasis, epithelial cells were found in peripheral blood. The number of epithelial cells was significantly larger in patients with extensive disease.

The assay was further configured to an image cytometric analysis. Using a fluorescence-based microscope image analysis system, visualization of events are easily obtain and the assessment of morphologic features to further identify objects is possible.

The CellTracks® System refers to an automated fluorescence microscopic system for automated enumeration of isolated cells from blood. The system contains an integrated computer controlled fluorescence microscope and automated stage with a magnetic yoke assembly that will hold a disposable sample cartridge. The magnetic yoke is designed to enable ferrofluid-labeled candidate tumor cells within the sample chamber to be magnetically localized to the upper viewing surface of the sample cartridge for microscopic viewing. Software presents suspect cancer cells, labeled with antibodies to cytokeratin and having epithelial origin, to the operator for final selection.

While isolation of tumor cells for the CellTracks® System can be accomplished by any means known in the art, one embodiment uses the Immunicon AutoPrep® System for isolating tumor cells using 7.5 ml of whole blood. Epithelial cell-specific magnetic particles are added and incubated for 20 minutes. After magnetic separation, the cells bound to the immunomagnetic-linked antibodies are magnetically held at the wall of the tube. Unbound sample is then aspirated and an isotonic solution is added to resuspend the sample. A nucleic acid dye, monoclonal antibodies to cytokeratin (a marker of epithelial cells) and CD 45 (a broad-spectrum leukocyte marker) are incubated with the sample. After magnetic separation, the unbound fraction is again aspirated and the bound and labeled cells are resuspended in 0.2 ml of an isotonic solution. The sample is suspended in a cell presentation chamber and placed in a magnetic device whose field orients the magnetically labeled cells for fluorescence microscopic examination in the CellTracks® System. Cells are identified automatically in the CellTracks® System and candidate circulating tumor cells presented to the operator for checklist enumeration. An enumeration checklist consists of predetermined morphologic criteria constituting a complete cell (see example 1).

The diagnostic potential of the CellTracks® System, together with the use of intact circulating tumor cells as a prognostic factor in cancer survival, can provide a rapid and sensitive method for determining appropriate treatment. Accordingly in the present invention, the apparatus, method, and kits are provided for the rapid enumeration and characterization of tumor cells shed into the blood in metastatic and primary patients for prognostic assessment of survival potential.

The methods of the invention are useful in assessing a favorable or unfavorable survival, and even preventing unnecessary therapy that could result in harmful side-effects when the prognosis is favorable. Thus, the present invention can be used for prognosis of any of a wide variety of cancers, including without limitation, solid tumors and leukemia's including highlighted cancers: apudoma, choristoma, branchioma, malignant carcinoid syndrome, carcinoid heart disease, carcinoma (i.e. Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell), histiocytic disorders, leukemia (i.e. B-cell, mixed-cell, null-cell, T-cell, T-cell chronic, HTLV-II-associated, lymphocytic acute, lymphocytic chronic, mast-cell, and myeloid), histiocytosis malignant, Hodgkin's disease, immunoproliferative small, non-Hodgkin's lymphoma, plasmacytolma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulose cell tumor, gynandroblastoma, hepatoma, hidradenoma, islet cell tumor, icydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophillia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondroscarcoma, cystosarcoma, phyllodes, fibrosarcoma, hemangiosarcoma, leiomyosarcoma, leukosarcoma, liposarcoma, lymphangiosarcoma, myoswarcoma, myxosarcoma, ovarian carcinoma, rhabdomyosarcoma, sarcoma (i.e. Ewing's experimental, Kaposi's and mast-cell), neoplasms (i.e. bone, breast, digestive system, colorectal, liver, pancreatic, pituitary, testicular, orbital, head and neck, central nervous system, acoustic, pelvic, respiratory tract, and urogenital, neurofibromatosis, and cervical dysplasia.

The following examples illustrate the predictive and prognostic value of CTC; CEC; or DTC from patients. Note, the following examples are offered by way of illustration and are not in any way intended to limit the scope of the invention.

### Example 1

### Circulating Endothelial Cells (CEC) and Circulating Tumor Cells (CTC) in Patients with Metastatic Colorectal Cancer

Lack of validated surrogate endpoints is an impediment to developing new cancer therapy. We hypothesized that CTC and CEC could predict outcome in pts undergoing treatment for metastatic colorectal cancer. Eligible patients for this multicenter study had metastatic colorectal cancer, and were initiating 1^{st}, 2^{nd}, or 3^{rd}-line systemic therapy. Blood was obtained at baseline and 3-4 weeks after treatment initiation for enumeration of CTC/CEC. CTC/7.5 ml and CEC/4 ml of blood were measured with the CellTracks® System. CTC were immunomagnetically enriched targeting CD326 (EpCAM), stained with DAPI, cytokeratin 8,18,19, and CD45. CEC expressing CD146 were immunomagnetically enriched and stained with DAPI, CD105, and counterstained with CD45. Cell morphology was confirmed in all cases.

In 139 controls CTC were virtually absent (0 CTC in 135 and 1 CTC in 4). For 131 pts with mCRC, >1CTC was detected before therapy in 40/131 (31%) (range 0-73) and 3-4 weeks after starting therapy in 12/131 (9%) (range 0-100) (p<0.0001, McNemar's test). Patients with >1 CTC at baseline and 3-4 weeks did not differ by line of therapy (1st line: 28/93 [30.1%] at baseline, 7/93 [7.5%] at 3-4 weeks; second/third-line: 6/24 [25%] at baseline, 3/24 [12.5%] at 3-4 weeks, p=0.57, Breslow-Day test). For 15/131 (11.5%) a 2-fold increase and for 53/131 (40.5%) a 2-fold decrease in CTC was found after treatment. Using 249 controls, a normal reference range of 4-80 CEC/4 ml of blood was established. In 16/131 (12.2%) patients >80 CEC were detected before (range 1-1342) and in 18/131 (13.7%) after start of therapy (range 2-519) (p=0.71, McNemar's test). Patients with >80 CEC at baseline and 3-4 weeks did not differ by line of therapy (1^{st} line: 9/93 [9.7%] at baseline, 13/93 [14%] at 3-4 weeks; 2^{nd}/3^{rd} line: 5/24 [20.8%] at baseline, 2/24 [8.3%] at 3-4 weeks, p=0.16, Breslow-Day test). In 37/131 (28.2%) a 2-fold increase and in 35/131 (26.7%) a 2-fold decrease in CEC was found after initiation of therapy.

Isolating and enumerating CTC/CEC in patients receiving therapy for metastatic colorectal cancer is feasible. CTC generally decrease with therapy, while change in CEC has greater variability.

### Example 2

### Circulating Endothelial Cells in Peripheral Blood of Healthy Subjects and Patients with Metastatic Carcinomas

In order to determine accuracy, precision, and linearity of endothelial cell enumeration in blood and compare CECs in healthy subjects and patients with metastatic carcinomas.

Blood was drawn in preservative tubes from controls and patients with metastatic carcinomas at multiple geographic locations. Samples were maintained at room temperature, shipped to a central laboratory, and processed within 72 hours of blood collection. All patients and healthy individuals were enrolled using approved protocols and provided informed consent. The healthy individuals used for comparison with the patients had no known illness or fever at the time of draw, no history of malignant disease, and were 35 years of age or older to provide a cohort age-matched with the cancer population.

The CellTracks® System (Immunicon, Huntingdon Valley, Pa.) used for endothelial cell enumeration consists of a CellTracks Autoprep®, an Endothelial Cell Kit and a CellSpotter™ Analyzer. The Endothelial Cell Kit consists of ferrofluids coated with CD146 antibodies to immunomagnetically enrich endothelial cells from 4 mL of blood. CD146 is expressed on endothelial cells, smooth muscle cells and a subset of activated T-lymphocytes. The enriched cells are labeled with the nuclear dye DAPI, CD105 conjugated to phycoerythrin and the pan-leukocyte antibody CD45 conjugated to allophycocyanin. CD105 is expressed on endothelial cells, activated monocytes and pre-B-lymphocytes^{x}. Buffers to wash, permeabilize and resuspend the cells are also included. The CellTracks AutoPrep® is a fully-automated sample preparation system. Briefly, 4 mL of blood is mixed with 10 mL of buffer, centrifuged at 800 g for 10 minutes, and placed on the CellTracks Autoprep®. The instrument aspirates the plasma/buffer layer and adds the ferrofluids. After incubation and subsequent magnetic separation the unbound cells and remaining plasma are aspirated. Next the staining reagents are added in conjunction with a permeablization buffer to fluorescently label the immunomagnetically bound cells. After incubation the magnetic separation is repeated to allow for the removal of excess staining reagents. In the final processing step, the cells are resuspended in the MagNest®, a magnetic cell presentation device (Immunicon, Huntingdon Valley, Pa.). This device consists of a chamber and two magnets that orient the immunomagnetically labeled cells for analysis on the CellTracks Analyzer. The MagNest is placed on the CellTracks Analyzer, a four color semi-automated fluorescent microscope. Image frames covering the entire surface of the cartridge for each of the four fluorescent filter cubes are captured. The captured images containing objects that meet predetermined criteria are automatically presented in a web-enabled browser from which final selection of cells is made by the operator. The criteria for an object to be defined as a CEC include variable morphology, a visible nucleus (DAPI positive), positive staining for CD105 and negative staining for CD45. Results of cell enumeration are expressed as the number of cells per 4 mL of blood. For enumeration, of CTCs the CellTracks system (Immunicon, Huntingdon Valley, Pa.) was used in combination with the Cell Search Tumor Cell Kit (Veridex, Warren, N.J.).

Assay performance was established with spiked HUVECs (human umbilical vein endothelial cell). The HUVEC cells were fluorescently labeled with DiOC16 before spiking to permit the discrimination of these cells from endogenous endothelial cells in the blood samples. To determine the linearity of the assay, 1280, 320, 80, and 20 HUVECs were spiked into 4 mL aliquots of blood from 5 different donors and processed for CEC enumeration. Assay precision was determined by spiking HUVECs labeled with DiOC16 at a concentration of ˜48 and ˜1040 cells into 4 mL aliquots of blood from one normal donor sample for 20 days. Samples were processed in duplicate with the same CellTracks Autoprep instrument. Lower level sensitivity was measured by spiking a very low number of DIOC16 labeled HUVEC (2 to 26 cells in 5 μL) onto the side of a CellTracks Autoprep tube. The exact cell number was counted on an inverted fluorescent microscope. 4 mL blood drawn was added to each Autoprep tube, mixed and processed on the CellTracks System. Recoveries of prelabeled cells were compared to each corresponding cell spike.

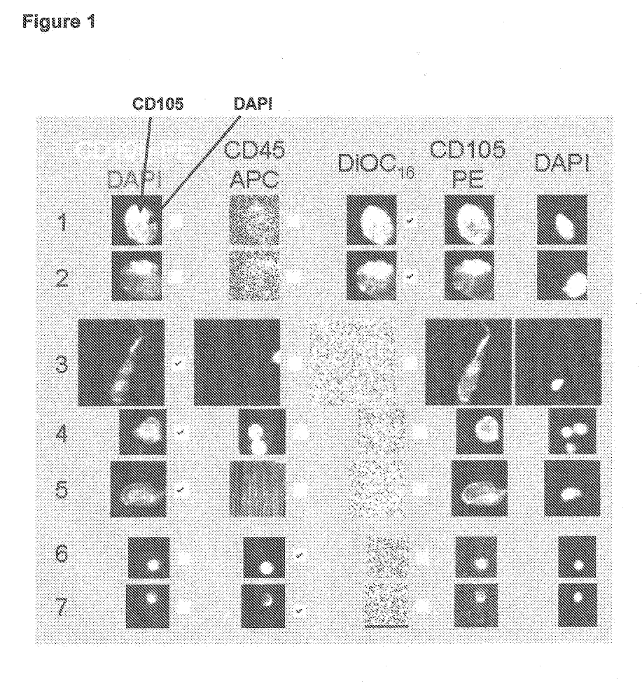

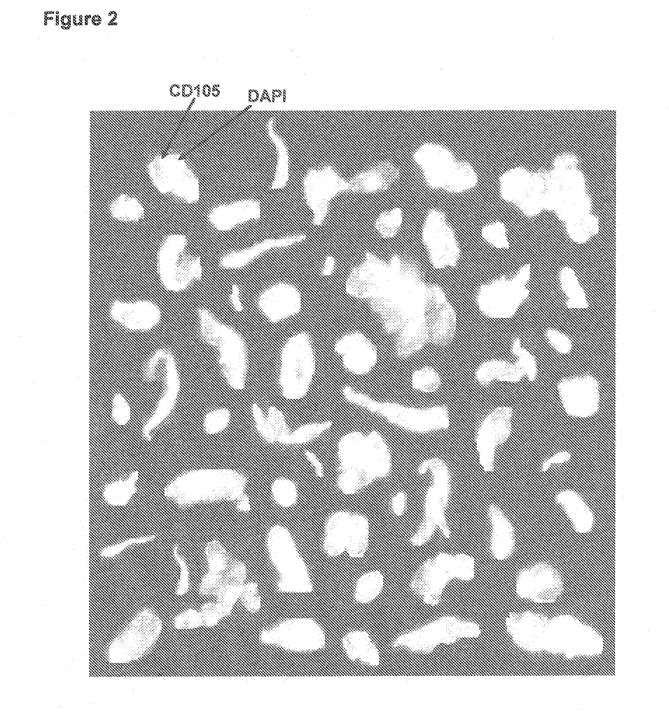

The CellTacks software identifies objects that stain with both DAPI and PE and displays these objects as thumbnails as is illustrated in FIG. 1. From right to left these thumbnails represent the nuclear (DAPI), CD105 PE, DiOC16 labeled HUVEC cells (DiOC16) and CD45 (CD45-APC) staining. The composite images shown at the left show a false color overlay of the nuclear (DAPI) and CD105-PE staining. Check boxes beside the composite, CD45-APC and DiOC16 image allow the user to confirm that the images represented in the row are consistent with endothelial cells, stain with the leukocyte marker CD45 or are the DiOC16 labeled HUVEC cells. The software tabulates the checked boxes for each sample and the information is stored in the database. In FIG. 1 thumbnails in row 1 and 2 illustrate DiOC16 labeled HUVEC cells identified by their staining with DAPI, CD105 and DiOC16 and lack of staining with CD45. Row 3, 4 and 5 show CECs identified by their staining with DAPI, CD105 and lack of staining with DiOC16 and CD45. Note that the endothelial cell presented in row 4 is surrounded by two leukocytes that stain with DAPI and CD45 but lack staining with DiOC16 and CD105. Row 6 and 7 show two cells staining with DAPI, CD105 and CD45 but not DiOC16. The latter cells are most likely leukocytes either specifically or non-specifically staining with CD105. Circulating Endothelial Cells have a typical morphologic appearance. FIG. 2 shows a gallery of typical CEC images.

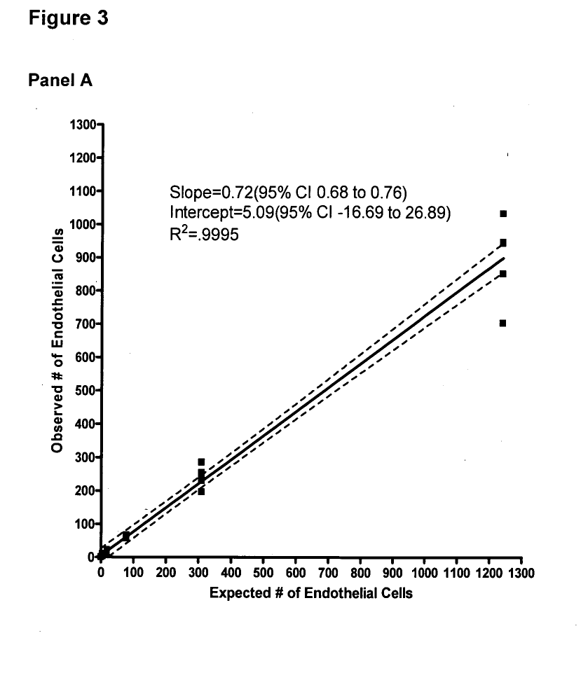

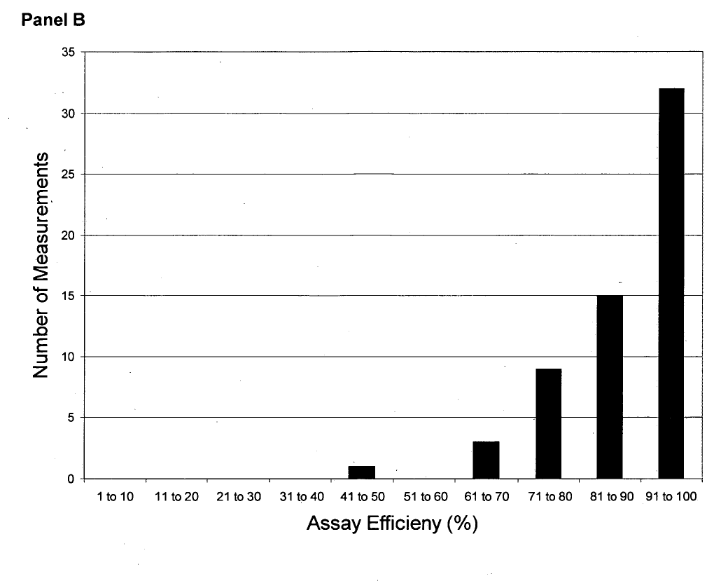

HUVEC cells were spiked into blood of five healthy donors at frequency of 5, 9, 78, 310 and 1241 cells and recovery was measured using the CellTracks system. In FIG. 3A, the approximate number of HUVEC cells spiked into the blood is plotted against the number observed in the samples. Regression analysis of the number of observed CECs versus the number of expected CECs resulted in a slope of 0.72 (95% confidence interval=0.68-0.76), an intercept of 5.1 (95% confidence interval=−17-27), and a correlation coefficient (R²) of 0.99. As expected, coefficient of variation (CV) increased as the number of cells spiked decreased, ranging from 12.5% at the 1241 cell spike to 37.3% at the 5 cell spike. The average HUVEC cells recovered was 85.6%.

The analytical lower limit of detection was measured by spiking a low number of DiOC16 labeled HUVEC cells in the sample processing tube and verification of the actual number of spiked cells under an inverted fluorescence microscope before addition of 4 mL of blood to the sample processing tube. The samples were processed and the spiked cells enumerated. The assay efficiency or percentage of spiked cells recovered was determined in 60 experiments and is illustrated in FIG. 3B. The cell spike ranged from 2 to 26 (mean 12, SD 5) and the recovery ranged from 44 to 100% (mean 86%, SD14).

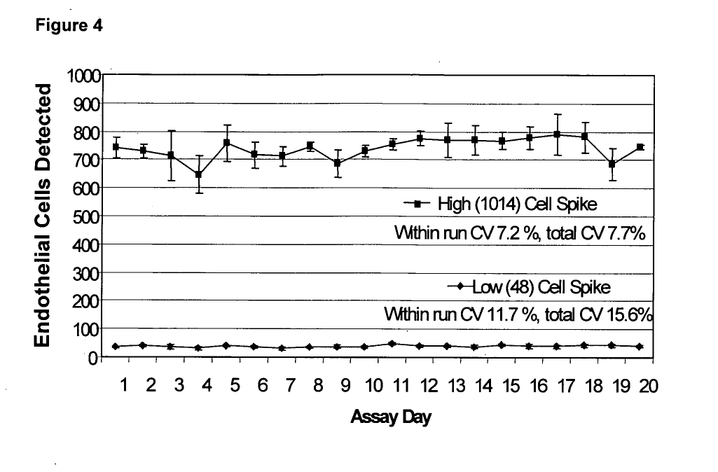

The reproducibility of CEC enumeration was measured using a single stock of DiOC16 labeled HUVEC cells spiked into blood from healthy donors at levels of 48 and 1014 cells/4 mL. Duplicate samples at each level were tested twice per day for 20 days and the results are shown in FIG. 4. The within run CV's for the 1014 cell spike and 48 cell spike were 7.2% and 11.7%, respectively. Similar results were found for total imprecision, with CV's of 7.7% and 15.6% for the 1014 and 48 cell spikes, respectively.

Blood from 15 healthy individuals was drawn into CellSave tubes and pooled. Aliquots of blood were made to evaluate CECs at 0, 24, 48, and 72 hours after blood draw. The difference in the number of CECs at 0 hours vs. 27, 48, and 72 hours was not significant (p=0.336, 0.198, and 0.666 respectively, paired t-test) demonstrating that blood samples drawn in CellSave tubes can be analyzed for CECs for up to at least 72 hours.

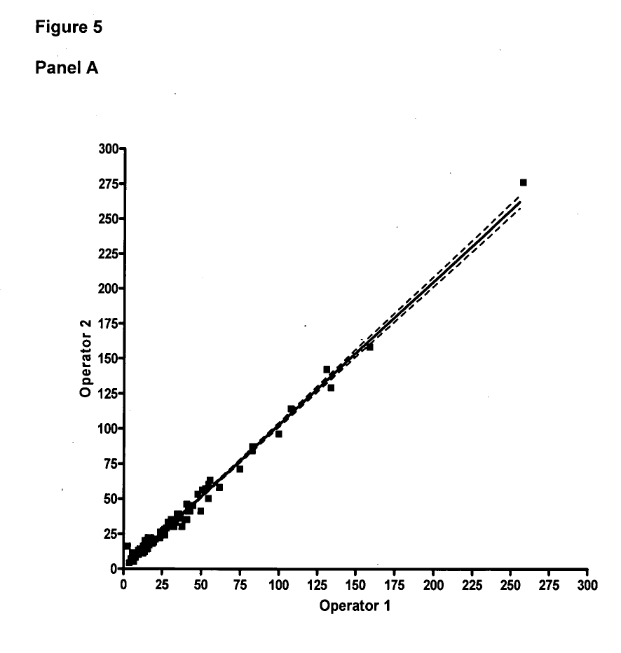

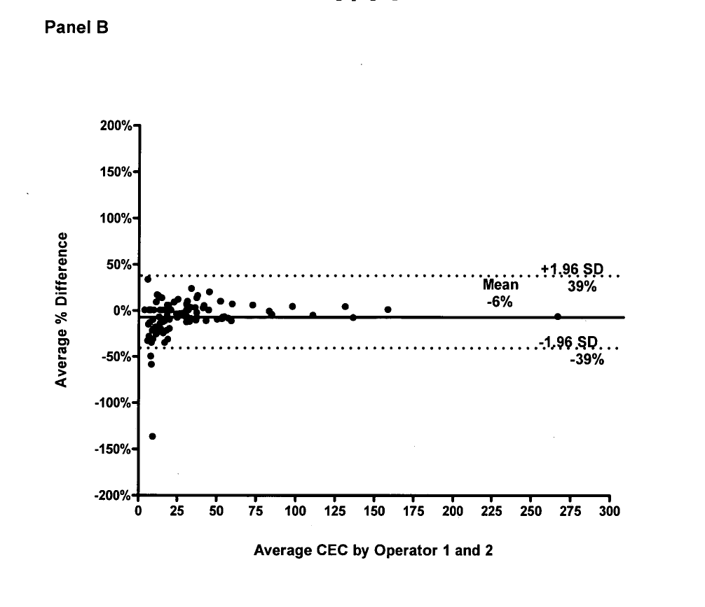

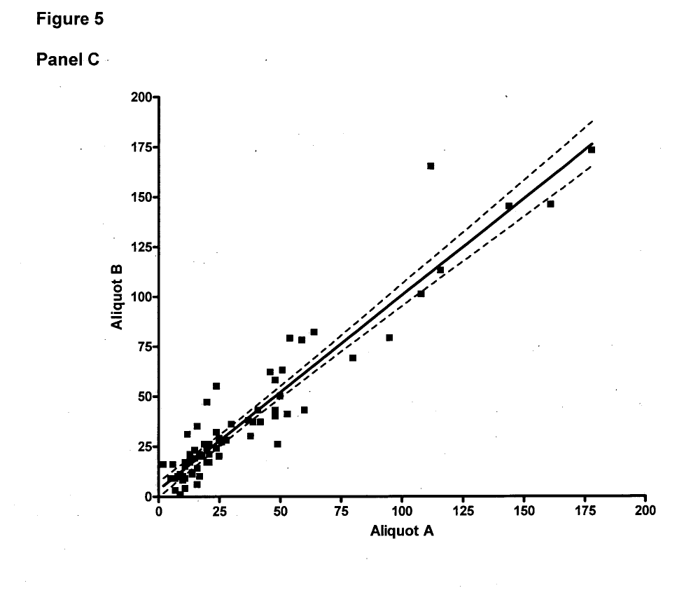

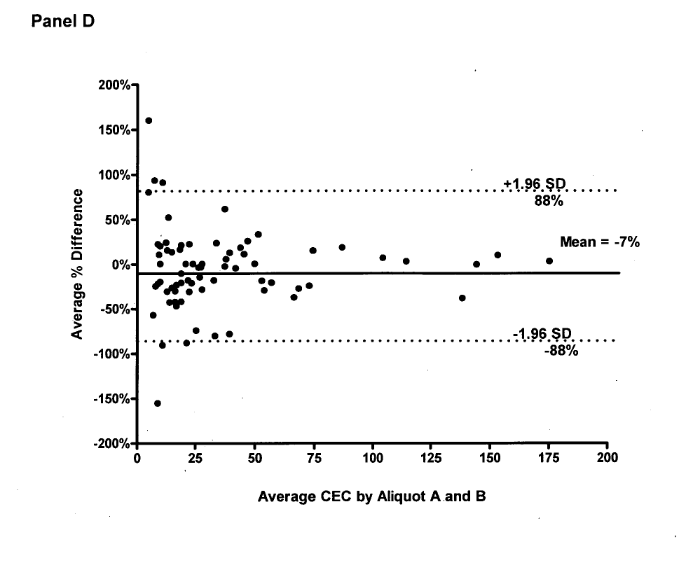

To measure the variability associated with the analysis of CECs, the browser images obtained from 100 blood specimens from healthy individuals and patients with various medical conditions were analyzed blindly by two different operators. Regression analysis demonstrated a slope of 1.03 (95% confidence interval 1.01 to 1.05), an intercept of 0.28 (95% confidence interval −0.72 to 1.29), and a correlation coefficient (R²) of 0.99. FIG. 5A, shows the correlation between both operators and FIG. 5B the data is shown using a Bland-Altman plot. The error of each CEC measurement is represented by the difference of the CEC count obtained by operator 1 is divided by the average of both CEC counts. This suggests that the criteria for selection of CECs from the images presented in the CellSpotter browser can be taught effectively.

To measure the variability in CEC counts obtained from two 4 mL aliquots derived from one evacuated blood draw tube CECs were enumerated in blood draw tubes from 72 different donors. Regression analysis showed a slope of 0.97 (95% confidence interval 0.89 to 1.05), an intercept of 3.55 (95% confidence interval −0.30 to 7.40), and a correlation coefficient (R²) of 0.90. FIG. 5C shows the correlation between both aliquots and FIG. 5D the data is shown using a Bland-Altman plot. The error of each CEC measurement is represented by the difference of the CEC count between both aliquots divided by the average of both CEC counts.

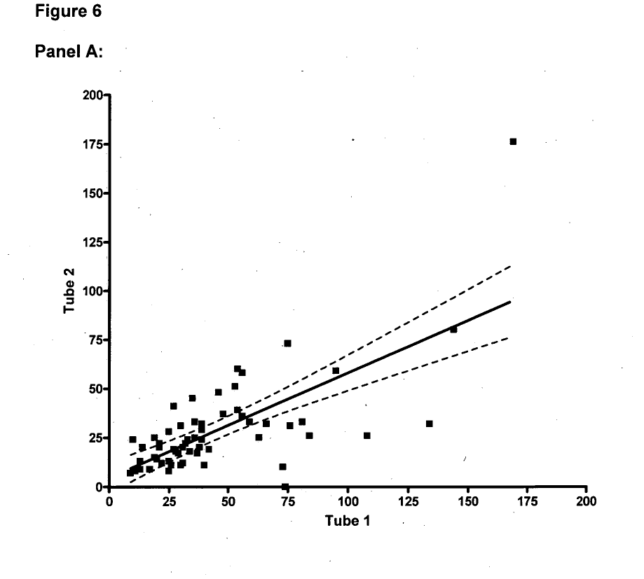

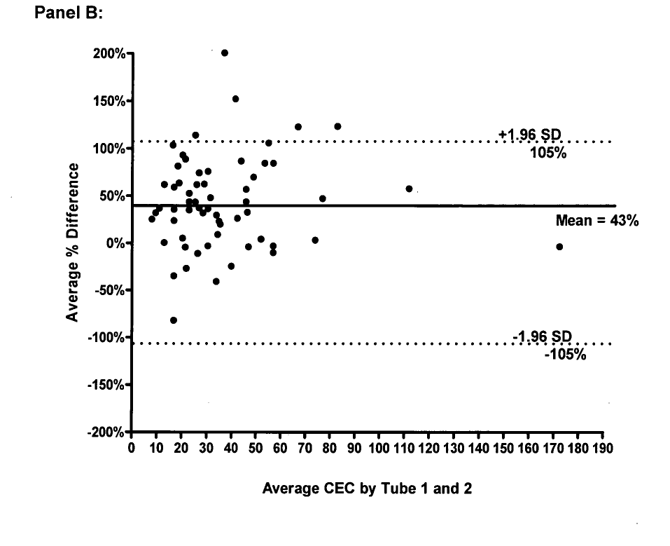

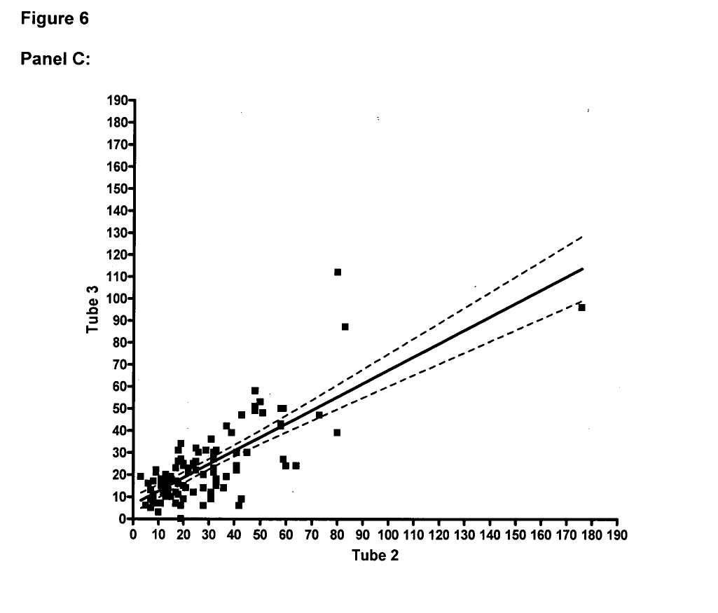

Endothelial cells were enumerated in different draw tubes to evaluate the effect of the vena puncture and the associated localized turbulence in blood flow on release of endothelial cells from the local vessel wall in the evacuated blood draw tube. Blood was collected from 66 healthy individuals and the CEC counts from the first tube were compared to those from the second tube. Regression analysis showed a slope of 0.53 (95% confidence interval 0.39 to 0.68), an intercept of 4.64 (95% confidence interval −3.31 to 12.59), and a correlation coefficient (R²) of 0.48. FIG. 6A shows the correlation between CECs in the first and second tube and FIG. 6B shows the corresponding Bland-Altman plot. The number of CECs in the first tube were significantly larger than those found in the second draw tube. To determine whether this increase in the number of CECs was due to the actual vena puncture blood was drawn from 100 donors and CECs were determined in the second and third blood draw tube. Regression analysis showed a slope of 0.61 (95% confidence interval 0.51 to 0.71), an intercept of 6.53 (95% confidence interval 2.82 to 10.24), and a correlation coefficient (R²) of 0.60. FIG. 6C shows the correlation between CECs in the first and second tube and FIG. 6D shows the corresponding Bland-Altman plot. The number of CECs in the second tube were not significantly larger than those found in the third draw tube. Although the correlation improved it did not reach the level obtained when CECs were enumerated in two aliquots of the same tube suggesting that not only the vena puncture but also the mere blood flow into the blood draw tube can release local endothelial cells in the draw tube. The correlation between CECs in the third versus later tubes from the same vena puncture did not further improve.

CECs were enumerated in blood samples from 167 healthy individuals and 206 patients with various metastatic carcinomas. FIG. 6 shows a scatter plot comparing CEC counts from healthy individuals and carcinoma patients and Table 1 summarizes the CEC counts. The distribution of the CEC counts were significantly different among healthy individuals and metastatic cancer patients (p-value=0.0001, Kruskal-Wallis Test). The median CEC counts were also significantly different between healthy individuals and metastatic cancer patients (p-value=0.000, Nonparametric k-sample test). The Fisher's exact test was used to demonstrate a statistically significant differences between the proportions of healthy volunteers and metastatic cancer patients with ≧100 CECs (p-value=0.000).

TABLE 1
Summary of CEC counts in 4 mL of blood from healthy individuals
and patients with various types of carcinomas.

| | No. of ≧Mean | | | | | | |
|---|---|---|---|---|---|---|---|
| | subjects | | | | ≧Mean | | |
| | 2SD (%) | Min | Max | Mean ± SD | Median + 1SD (%) | | |
| Healthy subjects | 167 | 0 | 916 | 28 ± 80 | 13 | 2 | 2 |

Metastatic Carcinoma

| | | | | | | | |
|---|---|---|---|---|---|---|---|
| Combined cancers | 206 | 0 | 1939 | 111 ± 255 | 34 | 23 | 11 |
| Breast Cancer | 50 | 1 | 471 | 78 ± 96 | 38 | 22 | 10 |
| Colon Cancer | 49 | 0 | 1375 | 86 ± 204 | 29 | 20 | 10 |
| Lung Cancer | 35 | 11 | 1546 | 146 ± 270 | 75 | 40 | 14 |
| Prostate Cancer | 48 | 3 | 1939 | 82 ± 279 | 27 | 10 | 4 |
| Other | 24 | 6 | 1499 | 240 ± 427 | 66 | 33 | 21 |

CECs and CTCs were enumerated in 124 metastatic carcinoma patients. CECs ranged from 6-1546 (mean 140 SD 274, median 50) per 4 mL of blood and CTCs ranged from 0-13254 (mean 112 SD 1190, median 0) per 7.5 mL of blood. Regression analysis showed no correlation between CECs and CTCs (R²=0.0012). Patients were separated into three clinically relevant groups those with 0 CTCs (n=91, 73%), those with 1 to 4 CTCs (n=15, 12%) and those with 5 or more CTCs/7.5 mL of blood (n=18, 15%). CECs for patients with 0 CTCs ranged from 6-1546 (mean 161±315, median 48). CECs for patients with 1-4 CTCs ranged from 13-297 (mean 87±82, median 79) and CECs for patients with 5 or more CTCs ranged from 14-246 (mean 80±72, median 54). The Kruskal-Wallis Test was used to test the equality of the CEC distributions within the three CTC groups and no significantly difference was found (p-value=0.93). The median CEC counts within the CTC groups were compared using the non-parametric k-sample test for equality of the medians and showed no significant difference (p-value=0.58). A nonparametric trend test was used to test for trends in CECs values within the CTC groups and were also not significantly different (p-value=0.81). Finally Spearman's Correlation was used to demonstrate that CECs within the CTC groups were independent (p-value=0.76).

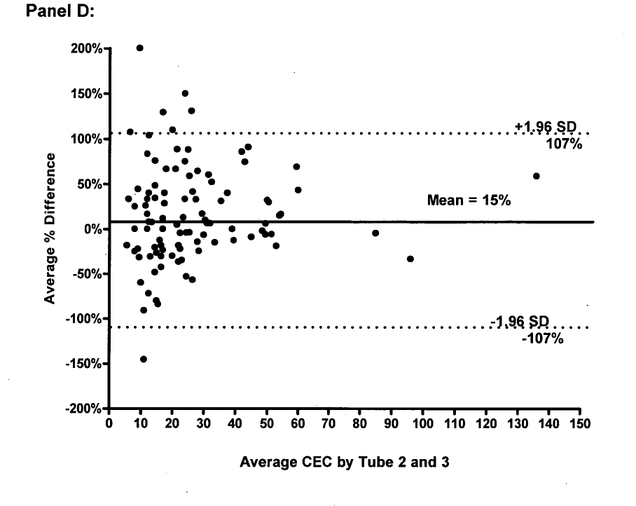

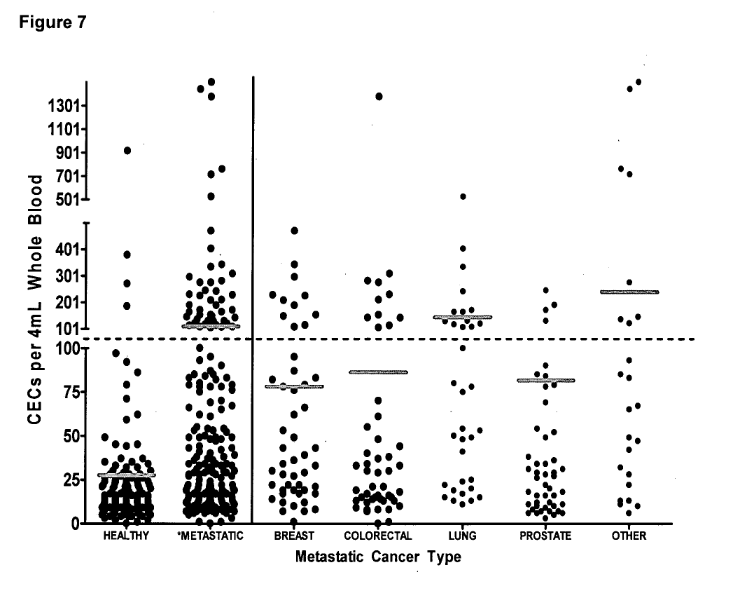

Endothelial cells play a key role in the development and growth of tumors. During this process endothelial cells may be released into the circulation. Enumeration and characterization of these circulating endothelial cells (CECs) may provide insights into the nature of specific disease processes and/or tumor response to treatment. Unfortunately, the frequency of CECs is low and, as current assay methods are inadequate, results can be highly variable. Hence, automation is needed to provide more consistent results. Therefore, we developed the CellTracks AutoPrep System—an automated system for rare cell sample preparation and analysis to include the analysis of rare CEC's. The system was used to determine the frequency of CECs in 4 mL of blood from healthy individuals and patients treated for metastatic carcinomas. In this system endothelial cells are defined as nucleated cells expressing S-endol/CD146, endoglin/CD105 and lacking the pan-leukocyte marker CD45. System accuracy and precision were validated using a model system employing cultured human umbilical vein cells (HUVEC) spiked into whole blood. The system precision was tested at low (50 cells) and high (1000 cells) cell spikes over 20 days. The system consistently recovered >70% of HUVEC cells from blood with a coefficient of variation of 7.5% for the high cell spike and 15.2% for the low cell spike. Recovery of HUVEC cells was linear over the tested range with a correlation R²=0.99. For enumeration of CECs different operators showed an excellent correlation (R²=0.99) in assigning cells as CECs and this correlation only slightly decreased when CECs were enumerated in two aliquots of the same blood draw tube (R²=0.90, FIG. 5). This correlation however decreased significantly when results were compared between the first and the second blood draw tube (R²=0.48) and improved only slightly from the second to the third blood draw tube (R²=0.61, FIG. 6). The higher number of endothelial cells found in the first collection tube may be explained by the release of endothelial cells due to the vena puncture. This however does not explain why the correlation between CECs in the second and third or later collection tubes does not improve the correlation to that obtained when two aliquots are analyzed from the same blood draw tube. Possible explanations are a release of endothelial cells from the local vessel wall due to the force introduced by the vacuum when a subsequent blood draw tube is introduced or a non random distribution of CECs in the blood. This observation stresses the need for markers that can identify the origin of the CECs. Markers that can identify CECs that originated from the capillary beds, large venous or arterial vessels or a specific organ would strongly enhance our understanding of CECs. Even though the correlation between CECs in different tubes drawn from the same vena puncture is not particular strong the number of CECs is elevated in a large portion of patients with metastatic carcinomas as compared to healthy individuals (Table 1, FIG. 7). Whether these CECs are derived from the tumor or are mere a results of damage of the vasculature due to therapy these patients are receiving is not known. Assessment of tumor associated antigen expression on CECs may allow the identification of CECs derived from the tumor whereas assessment of the age and viability status will further help in the characterization of CECs (Tem+PSMA+Nestin+apoptosis references).

No correlation was found between the number of CECs and Circulating Tumor Cells (CTCs) in the metastatic carcinoma patients suggesting two independent phenomenon. The presence of CTCs has been associated with poor prognosis (−) and the question arises what an elevated CEC count implies for patients with carcinomas.

The study demonstrates that CECs can be enumerated accurately and reproducibly. Following CECs during the course of the disease and various therapies is needed to determine their significance. Further differentiation of the phenotype of CECs will help to further elucidate vascular processes in patients treated for carcinomas.

### Example 3

### Peri-Operative Assessment of Circulating Tumor Cells in Blood, Disseminated Tumor Cells in Bone Marrow, and Tissue Gene Signatures in Patients with Primary Breast Cancer

Approximately 30% of the 200,000 women diagnosed annually with breast cancer will recur. Without a validated assay to identify these patients, all become candidates for adjuvant therapy. Both Real-time RT-PCR analysis of primary tissue and detection of disseminated tumor cells (DTC) in bone marrow by immunohistochemistry (IHC) purportedly aid in identifying these patients. This study demonstrates that the automated immunomagnetic fluorescent detection systems used to detect ‘circulating’ tumor cells (CTC) in blood could also be used to quantify DTCs in marrow. Incidence of CTCs, DTCs and gene signatures in matched specimens were also compared.

30 ml blood and 3 ml bone marrow specimens were collected in a preservative peri-operatively from 33 consented primary breast cancer patients stage 0-III. 31 healthy donors were used to determine CTC background in blood while a separate 51 healthy marrow donors served as DTC controls. Both blood and marrow specimens were prepared on the CellTracks Autoprep system. Using the CellSpotter Analyzer, cells expressing EpCAM were enriched and were counted as tumor cells if they were also nucleated, expressed Cytokeratin and lacked CD45. The OncoType Dx Multi-gene RT-PCR assay was used to analyze paraffin treated tissue.

0/31 control blood donors had CTCs while 6/33 (18%) patients had ≧2 CTC/30 ml blood (Range 2-8, mean 3.2/30 ml, 2.4SD) [Fisher's exact p-value=0.025]. 4/51 (8%) control marrow donors had ≧1 DTC/3 ml marrow (range 1-6, mean 4 DTC/3 ml, SD2.4) while 9/33 (27%) patients had ≧1 DTC/3 ml (range 1-29, mean 8 DTC/3 ml, SD11) [Fisher's exact p-value=0.028]. 2 patients (1 DCIS, 1 Stage I) had positive CTC and DTC counts. 2 patients still had DTCs (26, 29/3 ml) after neoadjuvant therapy. Patients with OncoType Dx recurrence scores 6-15 (low risk) also had no detectable CTCs and/or DTCs.

Immunomagnetic enrichment/imaging systems can be used to quantify DTCs. CTCs and DTCs may provide prognostic information complementary to gene expression profiling and increasing the accuracy of assessment of risk of recurrence in patients with primary breast cancer. The DTC method is being further validated by comparison to 'IHC bone marrow assay in a multi center international study.

While certain of the preferred embodiments of the present invention have been described and specifically exemplified above, it is not intended that the invention be limited to such embodiments. Various modification may be made thereto without departing from the spirit of the present invention, the full scope of the improvements are delineated in the following claims.

What is claimed is:

1. A method for evaluating metastatic potential in test subjects having circulating endothelial cells comprising:
a) obtaining a blood specimen from said test subject, said specimen comprising a mixed cell population suspected of containing said circulating endothelial cells;
b) enriching a fraction of said specimen, said fraction containing said circulating endothelial cells;
c) confirming structural integrity of said circulating endothelial cells to be intact; and
d) analyzing said intact circulating endothelial cells, wherein said analyzing correlates intact circulating endothelial cells enumeration of said test subject with said metastatic potential based upon a predetermined statistical association.

2. A method as claimed in claim 1, wherein said fraction is obtained by immunomagnetic enrichment, wherein said specimen is mixed with magnetic particles coupled to a biospecific ligand which specifically binds to said circulating endothelial cells, to the substantial exclusion of other populations and subjecting specimen-magnetic particle mixture to a magnetic field to produce a cell suspension enriched in magnetic particle-bound circulating endothelial cells.

3. A method as claimed in claim 1, wherein said structural integrity is determined by a procedure selected from the group consisting of immunocytochemical procedures, RT-PCR procedures, PCR procedures, FISH procedures, flowcytometry procedures, image cytometry procedures, and combinations thereof.

4. A method as claimed in claim 1, wherein said analysis is based upon a change in said intact circulating endothelial cell enumeration, said change being indicative of said metastatic potential.

5. A method as claimed in claim 1, wherein said metastatic potential is determined for said test subjects from the group consisting of metastatic breast cancer test subjects, metastatic prostate cancer test subjects, bladder cancer test subjects, metastatic colon cancer test subjects, and combinations thereof.

6. A method as claimed in claim 1, wherein said integrity of circulating endothelial cells is determined from a group consisting of positive nucleus, positive CD 146 antigen, negative CD 105 antigen, negative CD 45 antigen, and combinations thereof.

7. A peri-operative analysis method for assessing disease progression in a test subject, said analysis system comprising:
a) stabilizing cells in a biological specimen from said test subject wherein characteristic determinants of said cells are maintained;
b) enriching a fraction of said specimen, said fraction containing intact disseminated tumor cells;
c) confirming structural integrity of said intact cells; and
d) analyzing said intact cells to provide prognostic information, wherein enumeration with said metastatic potential is based upon a predetermined statistical association.

8. The rare cell analysis system of claim 7, wherein said prognostic information is determined from quantitative information obtained from a group consisting of disseminated tumor cells, circulating tumor cells, and combinations thereof.
